(19) United States
(12) Patent Application Publication
(10) Pub. No.: US 2026/0207463 A1
(43) Pub. Date: Jul. 23, 2026

(54) MELANIN AND NANO-LIGNIN BASED SUNSCREEN COMPOSITION, AND A METHOD OF PREPARATION THEREOF

(71) Applicant: AVISA MYKO, INC., San Francisco, CA (US)

(72) Inventors: Sushrut Sandeep BHANUSHALI, Thane (IN); Neha Sudhakar KANER, Thane (IN); Abhijeet Maruti SHIRSAT, Thane (IN); Swaraj Priyaranjan KUNAL, Thane (IN); Ravindra ADGULWAR, Thane (IN); Seshagiri RAGHUKUMAR, Dona Paula (IN)

(21) Appl. No.: 19/142,667

(22) PCT Filed: Jul. 12, 2023
(86) PCT No.: PCT/IB2023/057166

§ 371 Date: Jun. 24, 2025

(30) Foreign Application Priority Data
Dec. 24, 2022 (IN) 202211075187

(51) Int. Cl. A61K 8/72; A61Q 17/04

(52) U.S. Cl. CPC A61K 8/72; A61Q 17/04; A61K 2800/413; A61K 2800/5922; A61K 2800/805

(57) ABSTRACT

The present invention discloses a sunscreen composition comprising a soluble melanin in a range of 1% to 30% w/w and a first excipient; a nano lignin in a range of 1% to 50% and a second excipient; and one or more third excipient. The present invention also discloses a method for enhancing the solubility of melanin and preparation of nano lignin.

### TECHNICAL FIELD

The present disclosure relates to cosmetic compositions. More particularly, the present disclosure relates to sunscreen compositions comprising melanin and highly dispersible nano lignin with improved UV absorbance properties.

### BACKGROUND

Cosmetics can be defined as any article intended to be rubbed, poured, sprinkled, sprayed on, introduced into, or otherwise applied to, the human body for cleansing, beautifying, promoting attractiveness or altering the appearance, and includes any article intended for use as a component of cosmetic. Cosmetics have large number of varieties and type, and these are classified based on their application, and user type. Sunscreen is also one of the cosmetic products exclusively used as an anti-tan lotion that protects the skin from harmful UV radiations. Sunscreen also protects from skin burns and other damages caused by sunlight exposure.

With the increasing awareness with respect to sunburn, skin ageing, skin cancer, the use of sunscreen has tremendously increased. New photoprotective ingredients are being studied and produced continuously in response to the recent studies on their effects on skin against solar radiation. The sunscreen compositions available as of now, consist of active ingredients that are further divided into organic filters and inorganic UV filters. The organic filters absorb the UV rays whereas the inorganic filters such as titanium oxide and zinc oxide are known to reflect and scatter the UV rays. The sunscreen compositions also include UV filters such as oxybenzone, ethylhexyl methoxycinnamate, octocrylene etc.

Also, many available UV filters are not easily removed by common wastewater treatment techniques and have been found ubiquitously in wastewater and surface water. These have led to concerns about human safety. Laboratory studies have shown that many sunscreen ingredients, released into seawater through humans, have the potential to damage corals by causing toxicity, mortality, and bleaching, with photo exacerbation. Although yet to be sufficiently verified, there have been concerns about systemic toxicity in human beings, particularly endocrine, reproductive, and neurologic effects in human beings. In vitro studies have even suggested that some UV filters affect the hormones and increase the risk of breast cancer.

Melanin is a pigment found in animals, plants, fungi and bacteria. It can also be synthesized from melanin precursors, such as tyrosine. Melanin contributes to the tolerance of organisms with respect to high levels of ultraviolet rays and thus has the potentials for use in sunscreens. Even though melanin is found in a number of organisms, including bacteria and fungi, and can also be synthesized, it's utilization in sunscreens has remained a challenge because of its low solubility in water. Therefore, creating and producing melanin with high water solubilities is required for use in sunscreen.

Lignin is the second most renewable resource of biomass after cellulose, that can be used as a phenol substitute in the phenolic resin of the wood adhesive. However, the problem of poor dispersibility of the lignin in the adhesive is serious, and in addition, the lignin is insoluble in water, so that the application effect of the lignin is not ideal.

Further, it is known that nano lignin is an excellent antibacterial and antioxidative agent owing to its surface chemistry and morphology. Nano lignin shows enhanced antibacterial activity due to the lower molecular weight and higher phenolic content.

However, the existing method for preparing the nano lignin still has the problems of complex process, large energy consumption, large nano lignin particles, uneven particle size distribution and the like.

Hence, there is a need for development of a sunscreen composition with components that may result in high solubilities having high SPF and high UV absorbance capabilities and method for preparing the same.

### SUMMARY OF THE INVENTION

One aspect of the present disclosure provides a sunscreen composition. The sunscreen composition includes a soluble melanin in a range of 1%-30% w/w and a first excipient, a nano lignin in a range of 1% to 50% and a second excipient, and one or more third excipient.

In some aspect of the present disclosure, the solubility of melanin in water is enhanced by adding a salt selected from a group consisting of a sodium bicarbonate, calcium chloride, sodium chloride or a mixture thereof.

In some aspect of the present disclosure, the first excipient is selected from a group consisting of a diluent, humectant, or a mixture thereof.

In some aspect of the present disclosure, the diluent is selected from a group consisting of demineralized water, distilled water, deionized water, purified water or a mixture thereof.

In some aspect of the present disclosure, the humectant is selected from a group consisting of propylene glycol, dipropylene glycol, glycerol, butylene glycol or a mixture thereof.

In some aspect of the present disclosure, the second excipient is selected from a group consisting of a dispersing agent, an emulsifier, bodying agent, emollient or a mixture thereof.

In some aspect of the present disclosure, the dispersing agent is selected from a group consisting of polyglyceryl-4 oleate (and) polyglyceryl-6 oleate (and) polyhydroxystearic acid, C12-C15 alkyl benzoate, caprylic capric triglyceride, mineral oil or a mixture thereof.

In some aspect of the present disclosure, the emulsifier is selected from a group consisting of an octyldodecanol (and) octyldodecyl xyloside (and) PEG-30 dipolyhydroxystearate, stearic acid, glyceryl monostearate, ceteareth-25, ceteareth-20, Steareth-21, Oleth-10, sodium cetearyl sulfate or a mixture thereof.

In some aspect of the present disclosure, the bodying agent is selected from a group consisting of ceto stearyl alcohol, stearyl alcohol, cetyl alcohol or a mixture thereof.

In some aspect of the present disclosure, the humectant is selected from a group consisting of propylene glycol, dipropylene glycol, glycerol, butylene glycol or a mixture thereof.

In some aspect of the present disclosure, emollient is selected from a group consisting of an octyldodecyl oleate (and) octyldodecyl stearoyl stearate (and) polyhydroxystearic acid (and) octyldodecanol, mineral oil, vegetable oils, caprylic capric triglyceride, iso propyl myristate, isopropyl palmitate, octyldodecanol, C12-15 alkyl benzoate or a mixture thereof.

In some aspect of the present disclosure, the third excipient is a preservative is selected from a group consisting of phenoxyethanol (and) ethylhexylglycerin, DMDM hydantoin (and) methylchloroisothiazolinone (and) methylisothiazolinone, phenoxyethanol, caprylyl glycol, sodium benzoate, potassium sorbate or a mixture thereof.

In some aspect of the present disclosure, the soluble melanin is a carboxyl ester of melanin, O-methylated melanin, or an O-acetylated melanin.

In some aspect of the present disclosure, the soluble melanin is colored wherein colours are yellow, brown, orange or a mixture thereof.

In some aspect of the present disclosure, the sunscreen composition further comprises biopolymers and biomolecules to enhance UV absorbance.

In some aspect of the present disclosure, the biopolymers and biomolecules are selected from a group consisting of soy protein isolate, pea protein isolate, soy lecithin, sunflower lecithin, lignin sulfonate, an inulin, an inulin acetate, pectin, gadusol, xanthochrome or mixture thereof.

In some aspect of the present disclosure, the melanin is obtained from a group consisting of Gliocephalotrichum species, Cladosporium sp., Chaetomium sp., Curvularia spp., Agaricus spp., Aspergillus spp, and a Alternaria spp, Pseudomonas spp., Bacillus spp., Streptomyces spp. and Aeromonas spp.

In some aspect of the present disclosure, the melanin is obtained from a fungal strain MTCC 5489.

In some aspect of the present disclosure, the particle size distribution of nano lignin ranges from 20 nm to 110 nm.

In some aspect of the present disclosure, the sunscreen composition is in form of a gel, a cream, a lotion, a serum, a moisturizer, an ointment, a powder, an oil, a spray, or a mask.

Another aspect of the present disclosure provides a method for enhancing the solubility of melanin. The method includes cultivating a Gliocephalotrichum simplex at a temperature of 28° C.-32° C. to obtain a melanin. The method further includes harvesting the melanin to obtain a powder. The method further includes dissolving the obtained powder in a salt solution containing sodium chloride, calcium chloride and sodium bicarbonate to obtain a soluble melanin.

Another aspect of the present disclosure provides a method for preparation of nano-lignin. The method includes dissolving sieved kraft lignin in ethylene glycol to obtain a solution of lignin in ethyl glycol. The method further includes heating and stirring the lignin-glycol solution. The method further includes filtering the lignin-glycol solution under vacuum to remove debris and solid to obtain a pure lignin solution. The method further includes adding 0.1N of HNO₃ into the obtained pure lignin solution to obtain a mother liquor. The method further includes decanting the mother liquor and further centrifuging the obtained pellet to further obtain a centrifuged residue. The method further includes collecting and washing a centrifuged residue with distilled water to obtain washed nano lignin pellet and further centrifuging the washed nano lignin pellet. The method further includes collecting the lignin pellet post centrifugation and freeze-drying the nano-lignin pellet to obtain nano-lignin powder.

In some aspect of the present disclosure, the particle size distribution of the obtained nano lignin powder ranges from 20 nm to 110 nm.

Another aspect of the present disclosure provides a sunscreen composition. The composition includes a soluble melanin in a range of 1% to 30% w/w and a first excipient, a nano lignin in a range of 1% to 50% and a second excipient, and one or more third excipient obtained by a method that includes dissolving the melanin in a salt solution containing sodium chloride, calcium chloride and sodium bicarbonate to obtain a soluble melanin. The method further includes dissolving sieved kraft lignin in ethylene glycol and further adding 0.1N of HNO₃ into the obtained pure lignin solution and further centrifugation to obtain lignin. The method further includes preparing a phase A by dissolving soluble melanin in a range of 2% w/w of the composition in demineralized water followed by addition of propylene glycol in a range of 1.50% w/w of the composition to obtain the continuous phase. The method further includes preparing a phase B, by mixing 5% w/w of nano lignin with 21% w/w of octyldodecyl oleate (and) octyldodecyl stearoyl stearate (and) polyhydroxystearic acid (and) octyldodecanol, 0.50% w/w of polyglyceryl-4 oleate (and) polyglyceryl-6 oleate (and) polyhydroxystearic acid, 4% w/w of octyldodecanol (and) octyldodecyl xyloside (and) PEG-30 dipolyhydroxystearate and 3% w/w of Ceto Stearyl Alcohol to obtain the phase B. Furthermore, heating phase A and phase B to achieve a temperature of 70 degree Celsius. Lastly, adding phase A into phase B followed by addition of 0.50% w/w of phenoxyethanol (and) ethylhexylglycerin to obtain the sunscreen composition. In another aspect of the present disclosure, use of a sunscreen composition for anti-erythema is provided. The sunscreen composition includes a soluble melanin in a range of 1% to 30% w/w. The composition further includes a nano lignin in a range of 1% to 50% w/w. The composition further includes one or more of excipient selected from the group comprising of a solubility enhancer as sodium bicarbonate in a range of 0.5% to 2.5% w/w of the composition, a humectant as propylene glycol in a range of 1% to 5% w/w of the composition, an emollient as octyldodecyl oleate (and) octyldodecyl stearoyl stearate (and) polyhydroxystearic acid (and) octyldodecanol in range of 1% to 50% w/w of the composition, a dispersing agent as polyglyceryl-4 oleate (and) polyglyceryl-6 oleate (and) polyhydroxystearic acid in a range of 0.1% to 5% w/w of the composition; an emulsifier as octyldodecanol (and) octyldodecyl xyloside (and) PEG-30 dipolyhydroxystearate in a range of 1% to 5% w/w of the composition, a bodying agent as ceto stearyl alcohol in a range of 1% to 10% w/w of the composition and a preservative as phenoxyethanol (and) ethylhexylglycerin in a range of 0.5% to 1.11% w/w of the composition.

In another aspect of the present disclosure, a sunscreen composition with SPF 30 is provided. The sunscreen composition includes a phase A comprising soluble melanin in a range of 1% to 30% w/w and a first excipient. The sunscreen composition further includes a phase B comprising nano lignin in a range of 1% to 50% and a second excipient. The sunscreen composition further includes one or more third excipient. In some aspect of the present disclosure, the first excipient is selected from a group consisting of a diluent, humectant, or a mixture thereof.

In some aspect of the present disclosure, the second excipient is selected from a group consisting of a dispersing agent, emulsifier, bodying agent, humectant, emollient, or a mixture thereof.

In some aspect of the present disclosure, the third excipient is a preservative selected from a group consisting of phenoxyethanol (and) ethylhexylglycerin, DMDM hydantoin (and) methylchloroisothiazolinone (and) methylisothiazolinone, phenoxyethanol, caprylyl glycol, sodium benzoate, potassium sorbate or a mixture thereof.

In another aspect of the present disclosure, a sunscreen composition with SPF 55 is provided. The sunscreen composition includes a phase A comprising soluble melanin in a range of 1% to 50% w/w and a first excipient. The sunscreen composition further includes a phase B comprising xanthan gum in a range of 0.1% to 3% and a second excipient. The sunscreen composition further includes one or more third excipient.

### BRIEF DESCRIPTION OF DRAWINGS

The drawing/s mentioned herein disclose exemplary embodiments of the claimed invention. Other objects, features, and advantages of the present invention will be apparent from the following description when read with reference to the accompanying drawing:

FIG. 1 is a graphical representation that illustrates the effect of sodium chloride, calcium chloride and sodium bicarbonate on the solubility of melanin, according to an embodiment herein.

FIG. 2 illustrates the alteration in color of melanin by addition of cysteine, according to an embodiment herein.

FIG. 3 is a graphical representation that illustrates the effect of oxygenation on melanin UV absorption, according to an embodiment herein.

FIG. 4 depicts the chemical structure of ester of carboxylic acid of melanin with 1-Octanol.

FIG. 5 illustrates an NMR spectra of melanin and melanin ester with 1-Octanol, according to an embodiment herein.

FIG. 6 depicts a chemical structure of O-acetylated melanin ester, according to an embodiment herein.

FIG. 7 depicts a chemical structure of O-methylated melanin ester, according to an embodiment herein.

FIG. 8 illustrates discolourization and bleaching of melanin at neutral pH conditions, according to an embodiment herein.

FIG. 9 illustrates discolourization and bleaching of melanin in alkaline conditions, according to an embodiment herein.

FIG. 10 illustrates discolourization and bleaching of melanin, according to an embodiment herein.

FIG. 11 illustrates ozonation of melanin in water at pH 7, according to an embodiment herein.

FIG. 12 illustrates ozonation of melanin in water at pH 12, according to an embodiment herein.

FIG. 13 illustrates particle size distribution of nano-lignin, according to an embodiment herein.

FIG. 14 illustrates UV-visible absorbance spectrum of lignin, according to an embodiment herein.

FIG. 15 illustrates discolourization and bleaching of lignin, according to an embodiment herein.

### DETAILED DESCRIPTION OF PREFERRED EMBODIMENTS

This section is intended to provide explanation and description of various possible embodiments of the present invention. The embodiments used herein, and the various features and advantageous details thereof are explained more fully with reference to non-limiting embodiments illustrated in the accompanying drawing/s and detailed in the following description. The examples used herein are intended only to facilitate understanding of ways in which the embodiments may be practiced and to enable the person skilled in the art to practice the embodiments used herein. Also, the examples/embodiments described herein should not be construed as limiting the scope of the embodiments herein.

This section is intended to provide explanation and description of various possible embodiments of the present invention. The embodiments used herein, and the various features and advantageous details thereof are explained more fully with reference to non-limiting embodiments illustrated in the accompanying drawing/s and detailed in the following description. The examples used herein are intended only to facilitate understanding of ways in which the embodiments may be practiced and to enable the person skilled in the art to practice the embodiments used herein. Also, the examples/embodiments described herein should not be construed as limiting the scope of the embodiments herein.

As mentioned, there is a need for a sunscreen composition with high SPF and high UV absorbance properties.

“Melanin” refers to a large group of molecules responsible for biological functions such as pigmentation of hair and skin and further photoprotection of eyes and skin. The melanin is used as a skin protection, conditioning, and anti-ageing agent.

“Lignin” is an organic polymer which is found in cell walls of specific plant cells. The use of lignin in cosmetic composition provides a broad-spectrum sunlight protection as the lignin absorbs the harmful UV rays of sunlight and suppresses the formation of free radicals.

“Nano lignin” is an excellent antibacterial and antioxidative agent owing to its surface chemistry and morphology and shows enhanced antibacterial activity due to the lower molecular weight and higher phenolic content.

As used herein, “excipient” refers to inactive substances that serves as a medium for other active substances. They may include emulsifiers, bodying agents, humectant, solubility enhancers, dispersing agents and others that are known to a person skilled in art.

As used herein, “soluble melanin” refers to the melanin having enhanced solubility obtained by dissolving melanin in water containing sodium bicarbonate, calcium chloride and sodium chloride.

The term “nano lignin” and “highly dispersible nano lignin” are interchangeably used across the disclosure.

The lignin was procured from Gautam Zen International Pvt Ltd, Kolkata.

The present sunscreen composition includes melanin and highly dispersible nano-lignin that produces a synergistic effect where the melanin and highly dispersible nano lignin absorb the sunrays thereby providing a broad range of protection against the harmful rays of sun and prevent the DNA damage of cells from sun rays.

The sunscreen composition of the present invention includes a soluble melanin in a range of 1% to 30% w/w and a first excipient, a highly dispersible nano lignin in a range of 1% to 50% and a second excipient, and one or more third excipient.

In an embodiment, the first excipient is selected from a group consisting of an emulsifier, a dispersing agent, a humectant, a bodying agent, a solubility enhancer, a fusion agent, a diluent, and a preservative. In another embodiment, the first excipient is selected from a group comprising a diluent and humectant.

In another embodiment, the diluent is selected from a group consisting of demineralized water, distilled water, deionized water, and purified water or a mixture thereof.

In another embodiment, the humectant is selected from a group consisting of propylene glycol, dipropylene glycol, glycerol, butylene glycol or mixture thereof.

In another embodiment, the second excipient is selected from a group consisting of an emulsifier, a dispersing agent, a humectant, a bodying agent, a solubility enhancer, a fusion agent, a diluent, and a preservative.

In another embodiment, the second excipient may also be selected from a group consisting of an emulsifier, a dispersing agent, a humectant, a bodying agent, an emollient, or a mixture thereof.

In another embodiment, the emulsifier is selected from a group consisting of octyldodecanol (and) octyldodecyl xyloside (and) PEG-30 dipolyhydroxystearate, stearic acid, glyceryl monostearate, ceteareth-25, ceteareth-20, Steareth-21, Oleth-10, sodium cetearyl sulfate or mixture thereof.

In another embodiment, the humectant is selected from a group consisting of propylene glycol, dipropylene glycol, glycerol, butylene glycol or a mixture thereof.

In another embodiment, the dispersing agent is selected from a group consisting of polyglyceryl-4 oleate (and) polyglyceryl-6 oleate (and) polyhydroxystearic acid, C12-C15 alkyl benzoate, caprylic capric triglyceride, mineral oil or a mixture thereof.

In another embodiment, the bodying agent is selected from a group consisting of ceto stearyl alcohol, cetyl alcohol, stearyl alcohol or a mixture thereof.

In another embodiment, the emollient is selected from a group consisting of an octyldodecyl oleate (and) octyldodecyl stearoyl stearate (and) polyhydroxystearic acid (and) octyldodecanol, mineral oil, vegetable oils, caprylic capric triglyceride, iso propyl myristate, isopropyl palmitate, octyldodecanol, C12-15 alkyl benzoate or a mixture thereof.

In an embodiment, the third excipient is selected from a group consisting of an emulsifier, a dispersing agent, a humectant, a bodying agent, a solubility enhancer, a fusion agent, an emollient, a diluent, a preservative, or a combination thereof. In another embodiment, the third excipient is a preservative.

In another embodiment, the preservative is selected from a group consisting of phenoxyethanol (and) ethylhexylglycerin, DMDM Hydantoin (and) methylchloroisothiazolinone (and) methylisothiazolinone, phenoxyethanol, caprylyl glycol, sodium benzoate, potassium sorbate or a mixture thereof.

In an embodiment, the sunscreen composition of the present invention comprises soluble melanin. In another embodiment, the sunscreen composition of the present invention comprises carboxyl ester of melanin. In another embodiment, the sunscreen composition of the present invention comprises an O-methylated melanin. In another embodiment, the sunscreen composition of the present invention comprises an O-acetylated melanin.

In another embodiment, the sunscreen composition of the present invention is in the form of a cream. In another embodiment, the sunscreen composition is available in the form of a gel. In another embodiment, the sunscreen composition is available in form of a lotion. In another embodiment, the sunscreen composition is available as a spray.

In another embodiment, the sunscreen composition is available as a liquid. In another embodiment, the sunscreen composition is available as a toner. In another embodiment, the sunscreen composition is available as a serum.

In an exemplary embodiment, a sunscreen composition is provided. The sunscreen composition includes a phase A comprising melanin in a range of 1% to 30% w/w and a first excipient, a phase B comprising highly dispersible nano lignin in a range of 1% to 50% and a second excipient, and third phase comprising one or more third excipient. The presence of two UV blocking agents that is soluble melanin and nanolignin in two different phases provide higher SPF and thereby imparting more protection against UV-A, UV-B and UV-C rays.

In an exemplary embodiment, biomolecules and biopolymers are used to enhance the absorbance of UV radiation and improve the performance of the sunscreen composition SPF. The materials are selected from a group consisting of soy protein isolate, pea protein isolate, soy lecithin, sunflower lecithin, lignin sulfonate, an inulin, an inulin acetate, pectin, gadusol, xanthochrome, kynurenine, phytoene, phytofluene, carotenes, titanium dioxide, zinc oxide or mixture thereof.

In another embodiment, minerals are used to enhance the absorbance of UV radiation and improve the performance of the sunscreen composition SPF. The minerals are selected from a group consisting of titanium dioxide, zinc oxide, nano titanium dioxide and nano zinc oxide.

In another embodiment, sunscreen composition further includes a SPF booster. In another embodiment, the SPF booster is a microcrystalline cellulose.

In another embodiment, the sunscreen composition further includes a rheology modifier. In another embodiment, the rheology modifier is a xanthan gum.

In another embodiment, a sunscreen composition is provided that includes a soluble melanin and a first excipient, a highly dispersible nano lignin in a range of 1% to 50% and a second excipient, and one or more third excipient, obtained by a method by dissolving the melanin in a salt solution containing sodium chloride, calcium chloride and sodium bicarbonate in a melanin solution to obtain soluble melanin. The method further includes dissolving sieved kraft lignin in ethylene glycol and further adding 0.1N of HNO₃ into the obtained pure lignin solution and further centrifugation to obtain highly dispersible nano lignin. The method further includes preparing a phase A by dissolving soluble melanin in a range of 2% w/w of the composition in demineralized water followed by addition of propylene glycol in a range of 1.50% w/w of the composition to obtain the phase A. The method further includes preparing a phase B, by mixing 5% w/w of highly dispersible nano lignin with 21% w/w of octyldodecyl oleate (and) octyldodecyl stearoyl stearate (and) polyhydroxystearic acid (and) octyldodecanol, 0.50% w/w of polyglyceryl-4 oleate (and) polyglyceryl-6 oleate (and) polyhydroxystearic acid, 4% w/w of octyldodecanol (and) octyldodecyl xyloside (and) PEG-30 dipolyhydroxystearate and 3% w/w of Ceto Stearyl Alcohol to obtain the phase B. Furthermore, heating phase A and phase B to achieve a temperature of 70-degree celsius. Lastly, adding phase A into phase B followed by addition of 0.50% w/w of phenoxyethanol (and) ethylhexylglycerin to obtain the sunscreen composition.

In another embodiment, use of a sunscreen composition for anti-erythema is provided. The sunscreen composition includes a soluble melanin in a range of 1% to 30% w/w. The sunscreen composition further includes a highly dispersible nano lignin in a range of 1% to 50% w/w. The sunscreen composition further includes one or more of excipient selected from the group comprising of a solubility enhancer as sodium bicarbonate in a range of 0.5% to 2.5% w/w of the composition, a humectant as propylene glycol in a range of 1% to 5% w/w of the composition, an emollient as octyldodecyl oleate (and) octyldodecyl stearoyl stearate (and) polyhydroxystearic acid (and) octyldodecanol in range of 1% to 50% w/w of the composition, a dispersing agent as polyglyceryl-4 oleate (and) polyglyceryl-6 oleate (and) polyhydroxystearic acid in a range of 0.1% to 5% w/w of the composition; an emulsifier as octyldodecanol (and) octyldodecyl xyloside (and) PEG-30 dipolyhydroxystearate in a range of 1% to 5% w/w of the composition, a bodying agent as ceto stearyl alcohol in a range of 1% to 10% w/w of the composition and a preservative as phenoxyethanol (and) ethylhexylglycerin in a range of 0.5% to 1.11% w/w of the composition.

In another embodiment, a sunscreen composition with SPF 30 is provided. The sunscreen composition includes a phase A comprising soluble melanin in a range of 1% to 30% w/w and a first excipient. The sunscreen composition further includes a phase B comprising highly dispersible nano lignin in a range of 1% to 50% and a second excipient. The sunscreen composition further includes one or more third excipient.

In another embodiment, the first excipient is selected from a group consisting of a diluent, humectant or a mixture thereof.

In another embodiment, the second excipient is selected from a group consisting of a dispersing agent, emulsifier, bodying agent, humectant, emollient, or a mixture thereof. In another embodiment, the third excipient is a preservative selected from a group consisting of phenoxyethanol (and) ethylhexylglycerin, DMDM hydantoin (and) methylchloroisothiazolinone (and) methylisothiazolinone, phenoxyethanol, caprylyl glycol, sodium benzoate, potassium sorbate or a mixture thereof.

In another embodiment a sunscreen composition with SPF 55 is provided. The sunscreen composition includes a soluble melanin in a range of 1% to 50% w/w and a first excipient. The sunscreen composition further includes a xanthan gum in a range of 0.1% to 3% and a second excipient. The sunscreen composition further includes one or more third excipient.

In an embodiment, a method for enhancing solubility of melanin is provided. The method includes cultivating a Gliocephalotrichum simplex at a temperature of 28°-32° C. to obtain a melanin. The method further includes harvesting the melanin to obtain a powder. The method further includes dissolving the obtained powder in a salt solution containing sodium chloride, calcium chloride and sodium bicarbonate to obtain a solution of melanin.

In another embodiment, the melanin is dissolved in water at a high concentration of 1 to 30 percent.

In another embodiment, a method for altering the chemistry of the soluble melanin and converting them to carboxylate esters of melanin and O-acetylated melanin is provided.

The obtained melanin is soluble in non-polar solvents and can be used in the oil phase of a sunscreen composition.

In an embodiment, a method for improving the UV absorbance of melanin is provided. The increase in UV absorbance of melanin enhances its potential capability to provide higher SPF values.

In an embodiment, the melanin is obtained from fungi. In another embodiment, the fungi are selected from a group comprising, but not limited to, a Gliocephalotrichum sp., Cladosporium spp., Chaetomium spp., Curvularia spp., Agaricus spp., Aspergillus spp., and Alternaria spp.

In a preferred embodiment, the melanin is also obtained from the fungus MTCC 5489 deposited under the Budapest Treaty at the International Depository Authority of the Microbial Type Culture Collection, CSIR-Institute of Microbial Technology, Chandigarh, India.

In an embodiment, the melanin is obtained from bacteria. In another embodiment, the bacteria are selected from a group comprising but not limited to a Pseudomonas spp., Bacillus spp., Streptomyces spp. and Aeromonas spp.

In an embodiment, a method of preparation of highly dispersible nano lignin is provided. The method (300) includes dissolving (302) sieved kraft lignin with ethylene glycol to obtain a solution of lignin in ethylene glycol. The method (300) further includes heating (304) and stirring the lignin-glycol solution. The method (300) further includes filtering (306) the lignin-glycol solution under vacuum to remove debris and solid to obtain a pure lignin solution. The method (300) further includes, adding (308) 0.1N of HNO₃ into the obtained pure lignin solution to obtain a mother liquor. Further, decanting (310) the mother liquor and further centrifuging the obtained pellet to further obtain a centrifuged residue. Further, collecting (312) and washing the centrifuged residue with distilled water to obtain washed highly dispersible lignin pellet and further centrifuging (314) the washed nano lignin pellet. Furthermore, collecting (316) the highly dispersible lignin pellet post centrifugation. Lastly, the method (300) includes freeze-drying (318) the highly dispersible lignin pellet to obtain nano-lignin powder.

In another embodiment, the particle size distribution of obtained nano lignin powder ranges from 20 nm to 110 nm.

In an embodiment, the solvent is selected from a group comprising, but not limited to, an ethylene glycol, propylene glycol, diethylene glycol, ethylene glycol monobutyl ether (EGBE), and ethylene glycol and monomethyl ether (EGME).

In an embodiment, the high dispersible nano lignin pellet is obtained by freeze drying. In another embodiment, the highly dispersible nano lignin pellet is obtained by vacuum oven drying. In another embodiment, the highly dispersible nano lignin pellet is obtained by spray drying.

In an embodiment the acid is selected from a group comprising, but not limited to, a nitric acid, sulphuric acid, hydrochloric acid, acetic acid, and a glacial acetic acid.

In an embodiment, the highly dispersible nano lignin is in form of a powder. In another embodiment, the highly dispersible nano lignin is in form of a slurry. In another embodiment, the highly dispersible nano lignin is in form of crystals. In another embodiment, the highly dispersible nano lignin is in form of granules. In another embodiment, the highly dispersible nano lignin is in form of a pellet. In another embodiment, the highly dispersible nano lignin is in form of a paste.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to methods, and experimental conditions described, as such methods and conditions may vary.

### Example 1: Experiment for Improving Solubility of Melanin by Altering Culture Conditions

The fungus Gliocephalotrichum simplex MTCC 5489 was inoculated into 250 ml of a culture medium containing 10 g/L of glucose, 1 g/L of yeast extract, 10 g/L of peptone, 10 g/L of L-tyrosine and <1 g/L of trace elements and <0.025 ml of vitamins (referred as GPYET medium) and grown on a rotary shaker at 200 rpm for 5 days. Two sets of culture were prepared.

One set of culture was grown at 28° C. and further melanin was obtained by removing the fungal biomass by centrifugation and collecting the culture filtrate. The culture filtrate was spray-dried or freeze dried to obtain the melanin powder. Various concentrations of the melanin were dissolved in distilled water and solubility was analyzed by either filtering through a 0.45-micron filter or by microscopically examining for particles.

Another set of culture was grown at 25° C. Melanin was obtained by removing the fungal biomass by centrifugation and collecting the culture filtrate. The culture filtrate was spray-dried or freeze dried to obtain the melanin powder. Various concentrations of the melanin were dissolved in distilled water and solubility was analyzed by either filtering through a 0.45-micron filter or by microscopically examining for particles.

Observation: This fungus secretes the water-soluble melanin pigment into the culture medium. The melanin can be obtained in a powdered form from the broth and can be used directly as a powder, or after converting the melanin into nanoparticles or as a solution, or in combinations.

Results: Melanin obtained from cultures grown at 28° C. dissolved up to 4 percent (w/v) in distilled water, while melanin obtained from cultures grown at 25° C. dissolved up to 30 percent (w/v) in distilled water.

### Example 2: Experiment for Improving Solubility of Natural Melanin by Addition of Salts

The fungal culture, designated MTCC 5498 was grown at 28° C. as in Example 1. Melanin obtained from the process was soluble at 4 percent (w/v). The solubility was further enhanced as follows.

0.5, 1.0, 1.5, 2.0 and 2.5 percent concentration of sodium chloride (w/v) was prepared by dissolving in distilled water. Further, increasing amounts of melanin were added and solubility was assessed.

The steps were repeated to prepare different concentrations of calcium chloride and sodium bicarbonate, respectively.

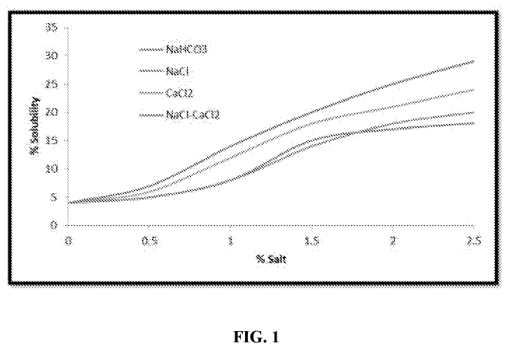

Results: It was found that dissolving melanin in solutions containing the sodium chloride, calcium chloride and sodium bicarbonate as above increased the solubility of melanin. A maximum solubility of 17.5 percent (w/v) was achieved with 2.5 percent sodium bicarbonate, 20 percent (w/v) with 2.5 percent sodium chloride, 25 percent (w/v) with sodium chloride and with 30 percent (w/v) with a combination of sodium chloride and calcium chloride. The results are depicted in FIG. 1

### Example 2: Experiment to Alter the Color of Natural, Soluble Melanin

MTCC 5498 was inoculated in 250 ml GPYET medium in a 1000 ml flask and grown on a shaker. Post 48 hrs of growth, three flasks were prepared wherein flask 1 had only fungal culture, flask 2 had 25 mM cysteine with fungal culture and flask 3 had 50 mM of cysteine and fungal culture.

Observation: Flasks without addition of cysteine were kept as control. The culture broth turned brick red to green after 96 hours and turned reddish green by 144 hours. Culture broth of control flasks without melanin were black in color.

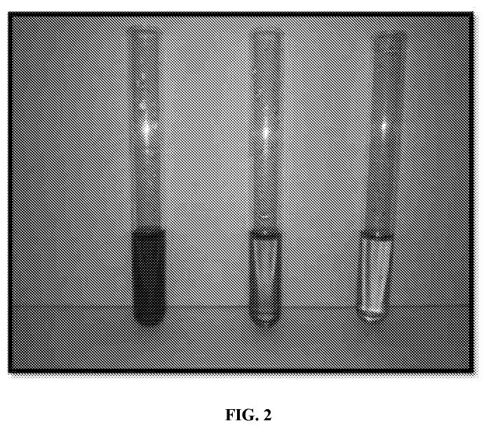

Results: The culture filtrate was separated after removing the biomass by filtration and freeze-dried. Control flasks without melanin produced 9.5 g dry weight of black melanin per liter culture medium. Flasks that received 25 mM of cysteine produced 13.9 g dry weight per liter of culture medium and those that received 50 mM cysteine produced 16.1 g dry weight melanin per liter culture medium. The results are represented in FIG. 2.

### Example 3: Increasing the Absorbance Capability of Soluble Melanin, Effect of Oxygenation on Melanin UV Absorption

The fungus was inoculated in a production medium containing 10 g/L of glucose, 10 g/L of peptone, 1 g/L of yeast extract, and 10 g/L of tyrosine along with zinc, manganese and copper salts. The experiment was further conducted in three flasks wherein each flask was provided sterile oxygen using 0.22-micron filter. The flasks were further transferred to incubator shaker at 25° C. and 200 rpm for 168 hrs. The oxygen was provided for a period of 60 hours (from 50 hrs-110 hrs after inoculation). The culture was harvested after 168 hours followed by centrifugation at 8000 rpm for 30 minutes. The supernatant was further freeze dried to get powdered melanin.

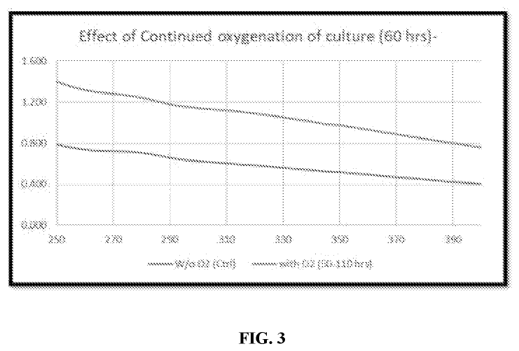

Result: A 0.01% w/v aqueous solution (neutral pH) of powdered melanin resulted in significant increase in UV absorption. The results for the same are depicted in FIG. 3.

### Example 4: Preparation of Melanin Derivatives

### Example 4.1: Preparation of Carboxylate Esters of Melanin

In a typical synthesis procedure, 50 grams of soluble melanin was taken in a round bottom flask. 600 ml of 1-Octanol was added to the flask and stirring was maintained at 400-500 rpm. The flask was fitted with a condenser tube and a dean-stark apparatus to extract the water out of the reaction mixture. The reaction flask was then heated to 100° C.-180° C. while stirring, using a heating mantle. Three to five mL concentrated sulfuric acid (H₂SO₄) was added as a catalyst to the flask, and 1-2 mL N, N-dimethyl formamide (DMF) was added as a co-catalyst. The reaction was carried out for 6 to 8 hours, following which the heating was stopped and the reaction mixture was allowed to cool down to room temperature. Further, water was collected in the dean stark apparatus, which confirmed the esterification and ensured that the ester does not get hydrolyzed. Further, the cooled reaction mixture was filtered using a Buchner funnel assembly, under vacuum. The solid was washed multiple times with methanol to ensure complete removal of residual 1-Octanol and sulfuric acid. The obtained material had a fine powder appearance after drying. The dried powder was characterized using FTIR and NMR spectroscopy to confirm the formation of ester.

The reaction was repeated with different alcohols, viz 2 ethyl 1-hexanol, isoamyl alcohol; isopentanol in place of 1-octanol using the same process.

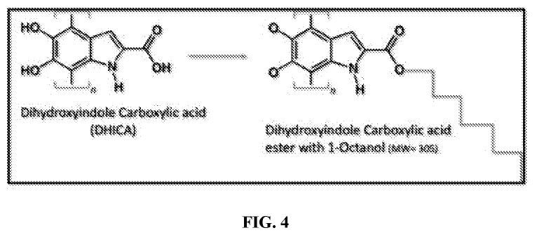

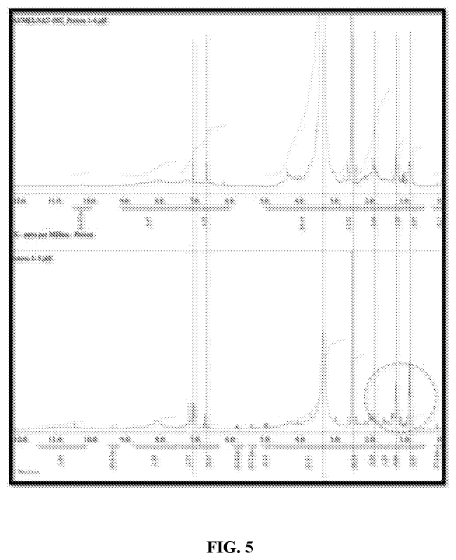

Result: The natural melanin was soluble in water, but insoluble in all organic solvents. The melanin ester was found to be insoluble in water, but soluble in dimethyl sulfoxide (DMSO), N, N-Dimethyl formamide (DMF) and N-methyl pyrrolidone (NMP). The structure is depicted in FIG. 4. The results for characterization using FTIR and NMR spectroscopy to confirm the formation of ester are presented in FIG. 5.

### Example 4.2: Preparation of O-Methylated Melanin

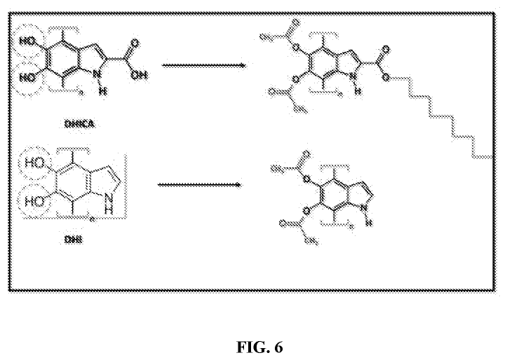

In a typical synthesis procedure, 48 g sodium hydroxide (NaOH) was dissolved in 200 ml distilled water. The solution was contained in a round bottom flask and stirred till a clear solution is obtained and allowed to cool to room temperature. To the NaOH solution, 50 grams of natural melanin was added and stirred using an overhead stirrer till the melanin is completely dissolved. The flask was fitted with a condenser tube and heated to 40-50° C. using a heating mantle. To the reaction mixture, 130 grams of dimethyl sulfate {(CH₃)₂SO₄} was added dropwise using a dropping funnel. The reaction was kept under further stirring for 1-2 hours post the addition of dimethyl sulfate, while maintaining the temperature at 50° C. The reaction mixture was allowed to cool down to room temperature (25° C.-30° C.). The reaction mixture was further neutralized by adjusting the pH at 7, by adding hydrochloric acid (HCl). The neutralized reaction mixture was filtered using a Buchner funnel assembly using a vacuum pump. The filtered product was washed with water successively. A clay like mass is obtained, which is freeze dried using a lyophilizer to obtain a fine, black powder. The powder was further stored in airtight container. The structure is depicted in FIG. 6.

### Example 4.3: Preparation of O-Acetylated Melanin

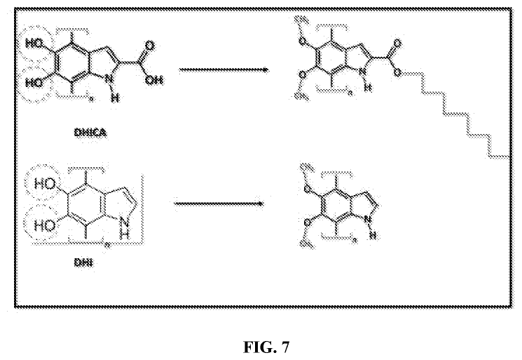

In a round bottom flask, 200 mL of ethylene dichloride (EDC) was taken, and 108 grams of anhydrous aluminum chloride (AlCl₃) was added stepwise while stirring. To the reaction mixture, 40 grams of melanin carboxylate ester obtained in example 9.2, was added while stirring. The reaction mixture was chilled to 5-10° C. using an ice bath. 64.5 grams of acetyl chloride was chilled separately to 0-5° C. and filled in a dropping funnel. The dropping funnel was connected to the above round bottom flask, and the acetyl chloride was added dropwise to the reaction mixture while stirring. The stirring was continued for 1-2 hours after the addition. The reaction mixture was allowed to warm up to room temperature and was further refluxed in EDC for 1-2 hours, at 80-90° C. The reaction mixture was allowed to cool to room temperature after refluxing. In a polypropylene plastic beaker, 1 kg of crushed ice and 100-150 ml of concentrated hydrochloric acid (HCl) were mixed. The ice-HCl mixture was stirred using a glass rod, and the reaction mixture was poured dropwise over the crushed ice to quench the reaction. Once the reaction quenched, the reaction mixture was filtered using a buchner funnel under vacuum. The product was washed multiple times with distilled water, followed by washing with methanol in the buchner funnel. The product was dried in the oven at 50° C.-60° C. to obtain a black colored powder. The powder was further stored in airtight container. The structure is depicted in FIG. 7.

### Example 5: Synthesis of Decolorized and Bleached Melanin

The process was carried out at two different pH ranges to achieve two different degrees of discoloration and/or bleaching of melanin.

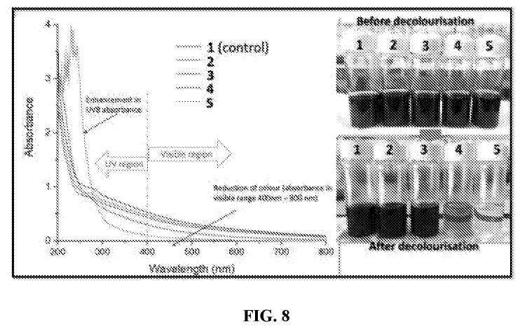

In the first process, natural melanin was dissolved in water at neutral pH (pH=6.5 to 7.5) at a concentration of 1% to 5% weight by weight. The solution was sonicated in an ultrasonicated for 30-90 minutes. Further, a bleaching agent was added, and the reaction vessel was closed. The concentration of the bleaching agent was maintained in the range of 0.1 weight percent to 25 weight percent, more specifically in the range of 1 wt. % to 15 wt. %. The solution was then stirred continuously for 3 hours to 48 hours. The concentration of bleaching agent and the duration of reaction was adjusted to achieve different degrees of decolorization and/or bleaching. At the end of the reaction, the reaction vessel was carefully opened to release the pressure generated by gases generated during the bleaching/decolorization action. The solutions were stored in refrigerator at 4° C. and are further freeze dried or alternatively lyophilized to obtain the decolorized and/or bleached product. The results for decolourization and bleaching of melanin at neutral pH conditions are depicted in FIG. 8.

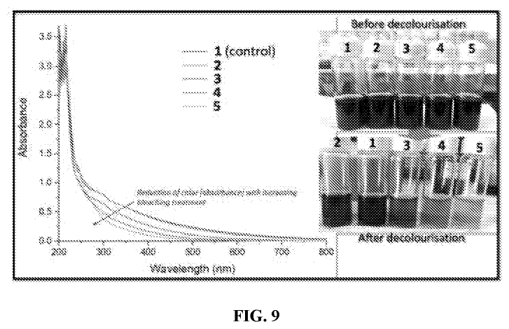

In the second process, 0.1-1 N of sodium hydroxide (NaOH) was prepared. Natural melanin is dissolved in the NaOH solution at alkaline pH at a concentration of 1% to 10% weight by weight. The solution was sonicated in an ultrasonicator for 30-90 minutes. Further, a bleaching agent was added, and the reaction vessel was closed. The concentration of the bleaching agent was maintained in the range of 0.1 weight percent to 25 weight percent (0.1-25 wt. %), more specifically 3.5 wt %. The solution was stirred continuously for 3 hours to 48 hours. The concentration of bleaching agent and the duration of reaction was adjusted to achieve different degrees of decolorization and/or bleaching. At the end of the reaction, the reaction vessel was carefully opened to release the pressure generated by gases during the bleaching/decolorization action. The solution was neutralized using dilute acid such as hydrochloric acid, sulfuric acid, nitric acid, acetic acid. The solution was stored in refrigerator at 4° C. and were further freeze dried or alternatively lyophilized to obtain the decolorized and/or bleached product. The results for discolourization and bleaching of melanin in alkaline conditions are depicted in FIG. 9.

Further, the bleaching agent can be selected from a group comprising, but not limited to, hydrogen peroxide (H₂O₂), sodium hypochlorite, and calcium oxychloride.

Furthermore, the aqueous alkaline solution can be selected from a group comprising, but not limited to, a potassium hydroxide, and sodium bicarbonate.

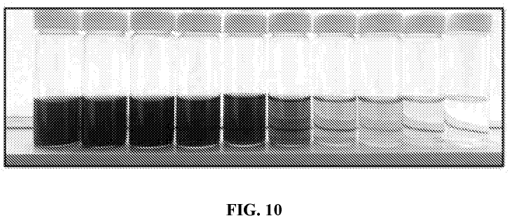

The result for discoloration and bleaching of melanin are depicted in FIG. 10, wherein the degree of decolorizations increases from left to right with the increasing concentration of bleaching agent and duration of reaction.

In another process, melanin was decolorized by way of ozonation and ozonolysis. In brief, 10 g of melanin was dissolved in 250 ml of water to form an aqueous solution. The pH was adjusted in the range of 7 to 12 using 1N NaOH. The obtained aqueous solution was contained in a hard glass container with closed cap and vent, and ozone gas was passed through the solution as microbubbles. The ozone gas was generated using POZ 1.0 ozonizer from TTK Prestige, with ozone production capacity of 200 mg/h. The degree of discoloration was controlled by the duration of ozonation in the range of 1 h to 24 h. The solution was freeze dried after ozonation to obtain decolorized melanin.

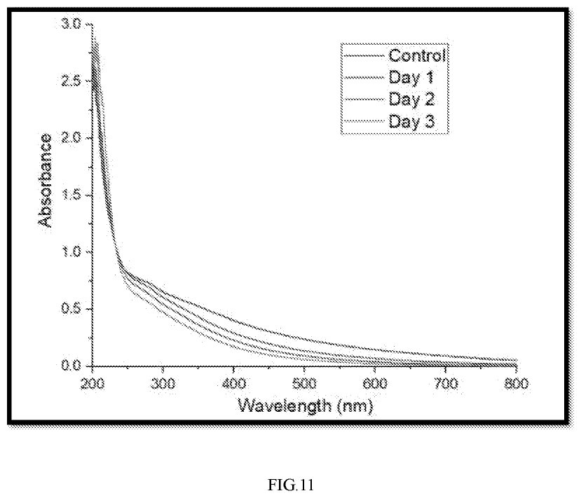

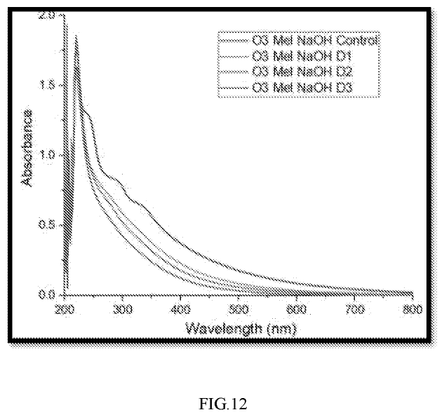

Observation: The ozonation step resulted in reduction of absorbance in the visible region (400-800 nm) of the spectrum, while retaining the absorbance in the UV region (200-400 nm) of the spectrum. The result for discoloration of melanin at pH 7 and pH 12 are depicted in FIG. 11 and FIG. 12.

### Example 6: Preparation of Highly Dispersible Nano-Lignin

In a typical synthesis, 5 grams of lignin powder (finely powdered, sieved) was added to 200 mL of ethylene glycol or propylene glycol. The lignin was solubilized by either heating and stirring and/or ultrasonicating the mixture for 30-60 minutes. The solubilized lignin was filtered using a filter paper in a Buchner funnel assembly under vacuum to remove the insoluble mass and debris (<1-2%) which can interfere with product quality if not removed. The filtered solution of lignin was kept for stirring on a magnetic stirrer at room temperature. 0.1 N solution of nitric acid (HNO₃) was prepared using distilled water and concentrated nitric acid. The dilute solution of nitric acid was filled in a dropping funnel or burette and was added dropwise to the lignin solution in ethylene glycol under agitation. The stirring was continued for 90-120 minutes post the addition of nitric acid is completed. The solution was kept standing overnight (12-18 hours) for settlement of the precipitated highly dispersible lignin. The mother liquor was decanted from the top as far as possible. The remainder of the solution was filled in multiple centrifuge tubes (of 50 mL) and were centrifuged at 8000-9000 rpm for 25-30 minutes. The supernatant was collected separately, and distilled water was added to the tubes containing the highly dispersible lignin pellet. The collected supernatant and decadent were reacidified with dilute nitric acid to have an additional crop of highly dispersible lignin. The highly dispersible lignin pellet was washed with the distilled water by shaking the tubes vigorously using a shaker and centrifuged again at 8000-9000 rpm for 25-30 minutes. At the end of the centrifugation, the pH of the supernatant was measured. The washing and centrifugation step was repeated 3-4 times, till the pH of the supernatant reaches pH 6-7. Once the pH reaches 6-7, the highly dispersible nano lignin pellet was collected, packed, and sealed.

Alternatively, hydrochloric acid, sulfuric acid or acetic acid was used for the acidification and precipitation in place of nitric acid.

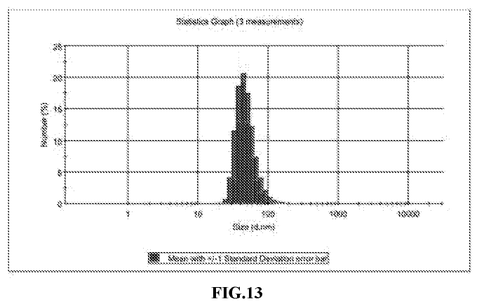

Result: The highly dispersible nano lignin pellet had a paste like consistency and has moisture content of 70-75%. The highly dispersible lignin paste was further freeze-dried using a lyophiliser/freeze drier to obtain a very fine, light cream-colored nano-lignin powder. This free-flowing powder was dispersible in oil continuous phase well as water phase. The overall yield of the process was 92-95% (by dry weight). The highly dispersible nano lignin was obtained which was recorded a mean particle size of 48 nm with a particle size distribution with 20 nm to 110 nm as depicted in FIG. 13.

### Results

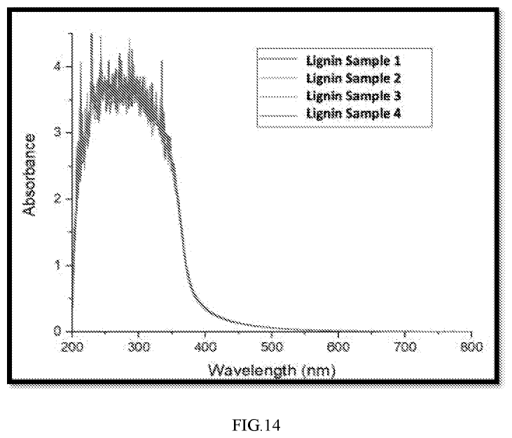

The results are depicted in FIG. 14, wherein UV-Visible absorbance spectrum of nano lignin was recorder in 4 different samples. The material exhibits minimal absorbance in the visible region of the spectrum (wavelength 400 nm-800 nm) and a high absorbance in the Ultraviolet (UVA and UVB) region of the spectrum (220 nm to 400 nm).

### Example 7: Synthesis of Decolorized and Bleached Highly Dispersible Nano-Lignin

### Example 7.1

The lignin was solubilized in 0.1 N NaOH. 10 grams of lignin was solubilized in 200 mL of NaOH solution and the pH was checked using pH paper, the pH was maintained in the range between 10 to 13. The solution was filtered through a filtration assembly consisting of buchner funnel, Whatman filter paper and vacuum flask, and vacuum pump. The debris collected on filter paper was discarded and the filtrate was used for discoloration. To the alkaline solution of lignin, hydrogen peroxide was added in a ratio of 0.1 to 10% v/v. The reaction vessel was closed, and the reaction mixture was stirred continuously for 3 hours to 24 hours, to achieve different degrees of discoloration. The discoloration action of the bleaching agent was ceased either by (a) dilution of the reaction mixture with water (b) warming of the reaction mixture in water bath in the temperature range from 50° C. to 80° C. (c) addition of a quenching agent to decompose the peroxide. After the bleaching, the solubilized lignin was precipitated out of the solution by acidification with 0.1-1 N nitric acid, hydrochloric acid or acetic acid. The pH was monitored, and when the pH drops to 6-7, the solution is allowed to stand for 3-24 hours. The flocculated precipitates of the lignin were seen to settle down in the solution. The solution was centrifuged at 3000-8000 rpm to separate the precipitate from the mother liquor. The mother liquor was further decanted, and the precipitate was washed thoroughly with deionized or distilled water and separated again by centrifugation. The wet precipitated lignin was allowed to dry either by (a) air drying in hot air oven at 50° C.-70° C., (b) vacuum drying at 30-50 degree celsius in vacuum oven (c) freeze drying/lyophilization. The fine, decolored lignin powder was obtained at the end of the process. The powder was found to form stable dispersions in aqueous as well as non-polar organic solvents.

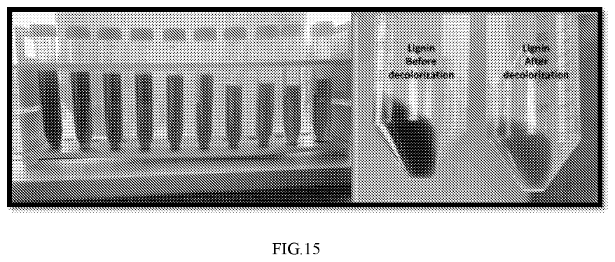

The results for synthesis of decolorized and bleached highly dispersible nano-lignin is depicted in FIG. 15, wherein the degree of decolorization increases from left to right with the increasing concentration of bleaching agent and duration of reaction.

Alternatively, the same process can be followed for bleaching with sodium hypochlorite and calcium oxychloride as the bleaching agent at 20° C.-60° C. temperature at pH ranges of 7-12.

### Example 7.2

In another approach the color of the lignin was reduced by way of ozonation to obtain decolorize lignin. In brief, 10 g of lignin was dissolved in 500 mL of 0.1N NaOH solution in water with pH 12. The solution was contained in a hard glass container with closed cap and vent, and ozone gas was passed through the solution as microbubbles. The ozone was generated using POZ 1.0 ozonizer from TTK Prestige, with ozone production capacity of 200 mg/h. The degree of discoloration was controlled by the duration of ozonation in the range of 1 h to 24 h. The solution was acidified using dilute nitric acid (HNO₃) till pH reached 2-3 to precipitate out bleached nanolignin. The precipitate was centrifugated at 7000-9000 rpm and washed 3 times with distilled water. The precipitate was further freeze dried to obtain fine powder of nanolignin.

### Example 7.3

In yet another approach for improving the absorbance of nanolignin, the as received Kraft lignin was dissolved in 500 mL of an organic solvent selected from a group, but not limited to, tetrahydrofuran, dioxane, or ethylene glycol. The solution thus obtained was ultrasonicated using an ultrasonication bath (Cole Parmer). The solution was filled in glass trays, and then exposed to UV radiation (5 W-10 W) for 1 h to 48 h. Further, the dissolved lignin was precipitated out as nanolignin using dilute aqueous solution (0.1N) of acid, from amongst nitric acid, hydrochloric acid, sulfuric acid, acetic acid. The precipitate was centrifugated at 7000 to 9000 rpm and washed several times with distilled water and freeze dried to obtain decolored nanolignin powder.

### Example 8: Static SPF Determination

The intrascapular area of the back to the right and left side of the midline of human volunteers was used for the experiment. Within this area, 30 cm² rectangular test sites were delineated with a gentian violet surgical skin marker. Sites were observed to ensure uniform pigmentation, skin tone and texture, and absence of warts, moles, nevi, scars, blemishes, and active dermal lesions. Any areas that might be expected to produce erratic results were not used for UV exposures.

The individual subject's Minimal Erythema Dose (MED) is the shortest time of exposure that produces minimally perceptible erythema at 16 to 24 hours post irradiation.

The compositions and 7% Padimate O/3% Oxybenzone standard were shaken and swirled with a glass rod before use and were evenly applied using plastic volumetric syringes or another device such as weigh boat or a spatula. The weighed powders or concentrated sprays were evenly applied to rectangular areas measuring a minimum of 30 cm* for a final concentration of 2.0 mg/cm*. Evenness of application was verified by observation with a Wood's Lamp.

Fifteen minutes post application, a protected site received a series of progressive UV light doses (minimum five) based upon previously determined MEDs. All immediate responses were recorded.

### Example 8.1 Evaluation of Responses

Sixteen to twenty-four hours post exposure, the subjects were instructed to return to the testing facility for evaluation of delayed erythemic responses. The spf was evaluated as follows:
SPF=Protected MED/Final unprotected MED
Visual grading scale:
0=No Erythema?
=Questionable Erythema 1=Minimal Erythema
2=Slight Erythema
3=Well-Defined Erythema 4=Erythema and Edema
5=Erythema and Edema in vesicles

The determination of all MEDs was carried out in a room with matte, neutral wall colors.

### Example: 8.2 Determination of the SPF Value

According to the reference, the mean SPF value (x) is calculated using a minimum of 10 evaluable subjects per composition/formulation. The standard deviation was determined(s). The upper 5% point was obtained from the t distribution table with n−1 degrees of freedom (t). The standard error (SE) was calculated by(s)/r′n (where n equals the number of subjects who provided valid test results).

Therefore, the label SPF value for panels using a minimum of 10 evaluable subjects is the largest whole number less than the mean SPF minus (t×SE).

Label SPF=Mean SPF−(t×SE)

### Example 9: Preparation of Sunscreen Emulsion Containing Soluble Melanin & Highly Dispersible Nano Lignin Powder

### Methodology: Preparation of Composition 1

### Phase A Preparation

2% w/w of soluble melanin was weighed and further mixed in 61% w/w of demineralized water with a glass rod in a glass beaker. Further, 2% w/w of sodium bicarbonate was added to the solution and mixed well. Further, 5.50% w/w of soluble melanin was added to the solution and stirred well to avoid formation of lumps. Further, 1.50% w/w of propylene glycol was added to the solution and mixed well.

### Phase B Preparation

In another glass beaker, 5% w/w of highly dispersible nano lignin was weighed and added followed by 12% w/w of octyldodecyl oleate (and) octyldodecyl stearoyl stearate (and) polyhydroxystearic acid (and) octyldodecanol and further mixed to prepare a uniform dispersion. Further, 3.50% w/w of polyglyceryl-4 oleate (and) polyglyceryl-6 oleate (and) polyhydroxystearic acid, 1-5% w/w of octyldodecanol (and) octyldodecyl xyloside (and) PEG-30 dipolyhydroxystearate and 1-10% w/w of Ceto Stearyl Alcohol was added to the mixture and mixed with a glass rod.

Further phase A and phase B were heated and continuously stirred with a glass rod till the temperature reaches 70 degree Celsius. Furthermore, phase A was added to phase B while under stirring. Lastly, 0.50% w/w of phenoxyethanol (and) ethylhexylglycerin was added to the mixture when the temperature reached 40 degrees Celsius.

TABLE 1
Composition 1

| Ingredients | % W/W |
|---|---|
| Phase A | |
| Demineralized Water | Up to 100% |
| Soluble Melanin | 7.50 |
| Sodium Bicarbonate | 2.00 |
| Propylene Glycol | 1.50 |
| Phase B | |
| Highly Dispersible Nano Lignin Powder (mean | 5.00 |
| particle size- 48 nm) | |
| Octyldodecyl Oleate (and) Octyldodecyl Stearoyl | 12.0 |
| Stearate (and) Polyhydroxystearic Acid (and) | |
| Octyldodecanol | |
| Polyglyceryl-4 Oleate (and) Polyglyceryl-6 Oleate | 3.50 |
| (and) Polyhydroxystearic Acid | |
| Octyldodecanol (and) Octyldodecyl Xyloside (and) | 4.00 |
| PEG-30 Dipolyhydroxystearate | |
| Ceto Stearyl Alcohol | 3.00 |
| Excipient | |
| phenoxyethanol (and) ethylhexylglycerin | 0.50 |

Result: The resultant in-vitro SPF for composition 1 was recorded as 30.19.

### Example 10: Preparation of Sunscreen Gel Containing High Absorbance Soluble Melanin

### Methodology: Preparation of Composition 2

### Phase A Preparation

Demineralized water and 2% w/w of high absorbance soluble melanin was added into a beaker and mixed well with a glass rod. Further, 2% w/w of Sodium Chloride was added into the mixture. Further, 13% w/w of melanin was added into the sample and mixed well.

### Phase B Preparation

1% w/w of Xanthan Gum and 3% w/w of Propylene Glycol were added into a petri dish. The samples were further mixed well with a spatula to form a uniform mixture. Furthermore, in a beaker, phase B was mixed with phase A followed by addition of. 0.50% w/w of phenoxyethanol (and) ethylhexylglycerin.

TABLE 2
Composition 2 (SPF 55)

| Ingredients | % W/W |
|---|---|
| Phase A | |
| Demineralized Water | Up to 100% |
| High Absorbance Soluble Melanin | 15.00 |
| Sodium Chloride | 2.00 |
| Phase B | |
| Xanthan Gum FF | 1.00 |
| Propylene Glycol | 3.00 |
| Excipient | |
| Phenoxyethanol (and) Ethylhexylglycerin | 0.50 |

Result: The in-vitro SPF for composition 2 was recorded as 55.76.

Further, a sunscreen gel containing natural melanin was prepared to show comparison from gel comprising highly absorbance melanin. The composition is depicted in Table 3 below.

TABLE 3

| Ingredients | % W/W |
|---|---|
| Phase A | |
| D. M. Water | 78.50 |
| Natural Melanin | 15.00 |
| Sodium Chloride | 2.00 |
| Phase B | |
| Xanthan Gum FF | 1.00 |
| Propylene Glycol | 3.00 |
| Phase C | |
| Phenoxyethanol (and) Ethylhexylglycerin | 0.50 |

Results: The composition resulted an in-vitro SPF of 29.71.

Thus, increasing the absorbance of melanin resulted in higher SPF values.

### Example 11: Preparation of Sunscreen Emulsion Containing High Absorbance Melanin & Nano Lignin

### Methodology: Preparation of Composition 3

### Phase A Preparation

In a beaker 50% w/w of demineralized water was added followed by addition of 10% w/w of high absorbance soluble melanin. The mixture was stirred with a glass rod to obtain a fine melanin solution. Lastly, 1.50% w/w of propylene glycol was added to the melanin solution.

### Phase B Preparation

In another glass beaker, 10% w/w/of nano lignin was added followed by addition of 17% w/w of light liquid paraffin. Nano lignin and light liquid paraffin were mixed with a glass rod to form a uniform dispersion. Further, 4% w/w of polyglyceryl-4 oleate (and) polyglyceryl-6 oleate (and) polyhydroxystearic acid and 4 5 w/w of octyldodecanol (and) octyldodecyl xyloside (and) peg-30 dipolyhydroxystearate were added o the dispersion and mixed well.

Furthermore, the mixture obtained in phase A and B were heated while maintaining constant mixing till a temperature of 70 degrees was achieved. Further, Phase A was transferred to phase B and stirred well to avoid formation of lumps. Lastly, 0.50% w/w of phenoxyethanol (and) ethylhexylglycerin was added into the mixture and further stirred to obtain a uniform mixture.

TABLE 4
Composition 3

| Ingredients | % W/W |
|---|---|
| Phase A | |
| D. M. Water | 50% |
| High absorbance Melanin | 10.00 |
| Propylene Glycol | 1.50 |
| Phase B | |
| Nano Lignin (mean particle size- 48 nm) | 10.00 |
| Light Liquid Paraffin | 17.0 |
| Polyglyceryl-4 Oleate (and) Polyglyceryl-6 | 4.00 |
| Oleate (and) Polyhydroxystearic Acid | |
| Octyldodecanol (and) Octyldodecyl Xyloside | 4.00 |
| (and) PEG-30 Dipolyhydroxystearate | |
| Ceto Stearyl Alcohol | 3.00 |
| Phase C | |
| Phenoxyethanol (and) Ethylhexylglycerin | 0.50 |

Result: The in-vivo SPF for composition 4 was recorded as 34.5.

### Example 12: Preparation of Sunscreen Emulsion Containing High Absorbance Soluble Melanin & Highly Dispersible Nano Lignin

### Methodology: Preparation of Composition 4

### Phase A Preparation

In a glass beaker, a 52% w/w of demineralized water was added followed by addition of 2% w/w of high absorbance soluble melanin and mixed well to obtain a melanin solution. 25% w/w of sodium bicarbonate was slowly added to the melanin solution and mixed well. Further, 5.50% w/w of melanin was added into the glass beaker and stirred to obtain a fine mixture. Lastly, 1.50% w/w of propylene glycol is added to the mixture and mixed well.

### Phase B Preparation

In a glass beaker, 5% w/w of highly dispersible nano lignin was added followed by addition of 21% w/w of octyldodecyl oleate (and) octyldodecyl stearoyl stearate (and) polyhydroxystearic acid (and) octyldodecanol and further mixed well using a glass rod to obtain a uniform mixture. Further, 0.50% w/w of polyglyceryl-4 oleate (and) polyglyceryl-6 oleate (and) polyhydroxystearic acid, 4% w/w of octyldodecanol (and) octyldodecyl xyloside (and) PEG-30 dipolyhydroxystearate and 3% w/w of Ceto Stearyl Alcohol was added to the mixture and mixed with a glass rod after addition of each component.

Furthermore, phase A and phase B were respectively heated to achieve a temperature of 70 degree Celsius. Both the phases were stirred while heating to avoid sedimentation. Further, phase A was added to phase B and stirred to mix well. Lastly, 0.50% w/w of phenoxyethanol (and) ethylhexylglycerin was added to the mixture and stirred till the temperature reached 40 degree Celsius.

### Result

TABLE 5
Composition 4 (SPF 30)

| Ingredients | % W/W |
|---|---|
| Phase A | |
| Demineralized Water | Up to 100% |
| High absorbance Soluble Melanin | 7.50 |
| Sodium Bicarbonate | 2.00 |
| Propylene Glycol | 1.50 |
| Phase B | |
| Highly Dispersible Nano Lignin (mean particle | 5.00 |
| size- 48 nm) | |
| Octyldodecyl Oleate (and) Octyldodecyl | 21.0 |
| Stearoyl Stearate (and) Polyhydroxystearic Acid | |
| (and) Octyldodecanol | |
| Polyglyceryl-4 Oleate (and) Polyglyceryl-6 | 3.50 |
| Oleate (and) Polyhydroxystearic Acid | |
| Octyldodecanol (and) Octyldodecyl Xyloside | 4.00 |
| (and) PEG-30 Dipolyhydroxystearate | |
| Ceto Stearyl Alcohol | 3.00 |
| Excipient | |
| Phenoxyethanol (and) Ethylhexylglycerin | 0.50 |

Result: The in-vivo SPF for composition 4 was recorded as 30 respectively.

### Example 13: Preparation of Sunscreen Emulsion Containing Bleached Melanin & Nano Lignin

### Methodology: Preparation of Composition 5

### Phase A Preparation

In a glass beaker, a 50.50% w/w of demineralized water was added followed by addition of 10% w/w of high absorbance soluble melanin and mixed well to obtain a melanin solution. 1.50% w/w of glycerin was slowly added to the melanin solution and mixed well. Lastly, 4% w/w of CI 77891 was added into the glass beaker and stirred to obtain a fine mixture.

### Phase B Preparation

In a glass beaker, 10% w/w of highly dispersible nano lignin was added followed by addition of 10% w/w of Paraffinum Liquidum and 4% w/w of polyglyceryl-4 oleate (and) polyglyceryl-6 oleate (and) polyhydroxystearic acid and further mixed well using a glass rod to obtain a uniform mixture. Further, 5% w/w of octyldodecanol (and) octyldodecyl xyloside (and) PEG-30 dipolyhydroxystearate 21% w/w of octyldodecyl oleate (and) octyldodecyl stearoyl stearate (and) polyhydroxystearic acid (and) octyldodecanol Further, 3% w/w of Ceto Stearyl Alcohol was added to the mixture and mixed with a glass rod after addition of each component.

Furthermore, phase A and phase B were respectively heated to achieve a temperature of 70 degree Celsius. Both the phases were stirred while heating to avoid sedimentation. Further, phase A was added to phase B and stirred to mix well. 1.50% w/w of poly isobutene was weighed and added when the temperature of 50-60 degree Celsius was achieved. Lastly, 0.50% w/w of phenoxyethanol (and) ethylhexylglycerin was added to the mixture and stirred till the temperature reached 40 degree Celsius.

### Result

TABLE 6
Composition 5 (SPF 51)

| Ingredients | % W/W |
|---|---|
| Phase A | |
| Demineralized Water | 50.50 |
| High absorbance bleached Melanin | 10 |
| Glycerin | 1.50 |
| CI 77891 | 4.00 |
| Phase B | |
| Highly Dispersible Nano Lignin (mean particle | 10.00 |
| size- 48 nm) | |
| Paraffinum Liquidum | 10.0 |
| Polyglyceryl-4 Oleate (and) Polyglyceryl-6 | 4.00 |
| Oleate (and) Polyhydroxystearic Acid | |
| Octyldodecanol (and) Octyldodecyl Xyloside | 5.00 |
| (and) PEG-30 Dipolyhydroxystearate | |
| Ceto Stearyl Alcohol | 3.00 |
| Excipient | |
| Poly isobutene | 1.50 |
| Phenoxyethanol (and) Ethylhexylglycerin | 0.50 |

Result: The in-vitro SPF for composition 5 was recorded as 51.84.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more aspects, configurations, or aspects for the purpose of streamlining the disclosure. The features of the aspects, configurations, or aspects may be combined in alternate aspects, configurations, or aspects other than those discussed above. This method of disclosure is not to be interpreted as reflecting an intention the present disclosure requires more features than are expressly recited in each claim. Rather, as the following claims reflect, inventive aspects lie in less than all features of a single foregoing disclosed aspect, configuration, or aspect. Thus, the following claims are hereby incorporated into this Detailed Description, with each claim standing on its own as a separate aspect of the present disclosure.

Moreover, though the description of the present disclosure has included description of one or more aspects, configurations, or aspects and certain variations and modifications, other variations, combinations, and modifications are within the scope of the present disclosure, e.g., as may be within the skill and knowledge of those in the art, after understanding the present disclosure. It is intended to obtain rights which include alternative aspects, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim:

1. A sunscreen composition, comprising:
a) a soluble melanin in a range of 1% to 30% w/w and a first excipient;
b) nano lignin in a range of 1% to 50% and a second excipient; and
c) one or more third excipient.

2. The sunscreen composition as claimed in claim 1, wherein the solubility of melanin in water is enhanced by adding a salt selected from a group consisting of sodium bicarbonate, calcium chloride, sodium chloride or a mixture thereof.

3. The sunscreen composition as claimed in claim 1, wherein the first excipient is selected from a group consisting of a diluent, humectant or a mixture thereof.

4. The sunscreen composition as claimed in claim 3, wherein the diluent is selected from a group consisting of a demineralized water, distilled water, deionized water, purified water or a mixture thereof.

5. The sunscreen composition as claimed in claim 3, wherein the humectant is selected from a group consisting of a propylene glycol, dipropylene glycol, glycerol, butylene glycol or a mixture thereof.

6. The sunscreen composition as claimed in claim 1, wherein the second excipient is selected from a group consisting of a dispersing agent, emulsifier, bodying agent, humectant, emollient or a mixture thereof.

7. The sunscreen composition as claimed in claim 6, wherein the dispersing agent is selected from a group consisting of polyglyceryl-4 oleate (and) polyglyceryl-6 oleate (and) polyhydroxystearic acid, C12-C15 Alkyl benzoate, caprylic capric triglyceride, mineral oil or a mixture thereof.

8. The sunscreen composition as claimed in claim 6, wherein the emulsifier is selected from a group consisting of an octyldodecanol (and) octyldodecyl xyloside (and) PEG-30 dipolyhydroxystearate, stearic acid, glyceryl monostearate, ceteareth-25, ceteareth-20, Steareth-21, Oleth-10, sodium cetearyl sulfate or a mixture thereof.

9. The sunscreen composition as claimed in claim 6, wherein the bodying agent is selected from a group consisting of a of a ceto stearyl alcohol, cetyl alcohol, stearyl alcohol or a mixture thereof.

10. The sunscreen composition as claimed in claim 6, wherein the humectant is selected from a group consisting of a propylene glycol, dipropylene glycol, glycerol, butylene glycol or a mixture thereof.

11. The sunscreen composition as claimed in claim 6, wherein the emollient is selected from a group consisting of octyldodecyl oleate (and) octyldodecyl stearoyl stearate (and) polyhydroxystearic acid (and) octyldodecanol, mineral oil, vegetable oils, caprylic capric triglyceride, iso propyl myristate, isopropyl palmitate, octyldodecanol, C12-15 alkyl benzoate or a mixture thereof.

12. The sunscreen composition as claimed in claim 1, wherein the third excipient is a preservative selected from a group consisting of phenoxyethanol (and) ethylhexylglycerin, DMDM hydantoin (and) methylchloroisothiazolinone (and) methylisothiazolinone, phenoxyethanol, caprylyl glycol, sodium benzoate, potassium sorbate or a mixture thereof.

13. The sunscreen composition as claimed in claim 1, wherein the soluble melanin is carboxyl ester of melanin, O-methylated melanin, or an O-acetylated melanin.

14. The sunscreen composition as claimed in claim 1, wherein the soluble melanin is colored wherein the colours are yellow, brown, orange or a mixture thereof.

15. The sunscreen composition as claimed in claim 1, wherein the sunscreen composition further comprises biopolymers and biomolecules to enhance UV absorbance.

16. The sunscreen composition as claimed in claim 15, wherein the biopolymers and biomolecules are selected from a group consisting of soy protein isolate, pea protein isolate, soy lecithin, sunflower lecithin, lignin sulfonate, an inulin, an inulin acetate, pectin, gadusol, xanthochrome or a mixture thereof.

17. The sunscreen composition as claimed in claim 1, wherein the melanin is obtained from a group consisting of, Gliocephalotrichum species, Cladosporium sp., Chaetomium sp., Curvularia spp., Agaricus spp., Aspergillus spp, and a Alternaria spp, Pseudomonas spp., Bacillus spp., Streptomyces spp. and Aeromonas spp.

18. The sunscreen composition as claimed in claim 1, wherein the melanin is obtained from a fungal strain MTCC 5489.

19. The sunscreen composition as claimed in claim 1, wherein the particle size distribution of nano lignin ranges from 20 nm to 110 nm.

20. The sunscreen composition as claimed in claim 1, wherein the composition is in form of a gel, a cream, a lotion, a serum, a moisturizer, an ointment, a powder, an oil, a spray, or a mask.

21. A method (200) for enhancing solubility of melanin, the method comprising:
a) cultivating Gliocephalotrichum simplex at a temperature of 28° C.-32° C. to obtain a melanin;
b) harvesting the melanin to obtain a powder; and
c) dissolving the obtained powder in a salt solution containing sodium chloride, calcium chloride and sodium bicarbonate to obtain a soluble melanin.

22. A method (300) for preparation of nano lignin, the method comprising:
a) dissolving (302) 0.5-5 gms of sieved kraft lignin in ethylene glycol to obtain a lignin glycol solution;
b) heating (304) and stirring the obtained lignin-glycol solution;
c) filtering (306) the lignin-glycol solution under vacuum to remove debris and solid to obtain a pure lignin solution;
d) adding (308) 0.1N of HNO₃ into the obtained pure lignin solution to obtain a mother liquor;
e) decanting (310) the obtained mother liquor and further centrifuging the obtained pellet to further obtain a centrifuged residue;
f) collecting (312) and washing the centrifuged residue with distilled water to obtain washed nano lignin pellet and further centrifuging (314) the washed nano lignin pellet;
g) collecting (316) the nano lignin pellet post centrifugation; and
h) freeze-drying (318) the nano-lignin pellet to obtain nano-lignin powder.

23. The method as claimed in claim 22, wherein the particle size distribution of the obtained nano lignin powder ranges from 20 nm to 100 nm.

24. A sunscreen composition, wherein the composition comprises:
a) a soluble melanin in a range of 1% to 30% w/w and a first excipient;
b) a nano lignin in a range of 1% to 50% and a second excipient; and
c) one or more third excipient;
obtained by a method comprising—
a) dissolving the melanin in a salt solution containing sodium chloride, calcium chloride or sodium bicarbonate to obtain a soluble melanin;
b) dissolving sieved kraft lignin in ethylene glycol and further adding 0.1N of HNO₃ into the obtained pure lignin solution;
c) centrifugation to obtain nano lignin;
d) preparing phase A by dissolving soluble melanin in a range of 2% w/w of the composition in demineralized water followed by addition of propylene glycol in a range of 1.50% w/w of the composition to obtain a phase A;
e) preparing phase B by mixing 5% w/w of nano lignin with 21% w/w of octyldodecyl oleate (and) octyldodecyl stearoyl stearate (and) polyhydroxystearic acid (and) octyldodecanol, 0.50% w/w of polyglyceryl-4 oleate (and) polyglyceryl-6 oleate (and) polyhydroxystearic acid, 4% w/w of octyldodecanol (and) octyldodecyl xyloside (and) PEG-30 dipolyhydroxystearate and 3% w/w of Ceto Stearyl Alcohol to obtain a phase B;
f) heating phase A and phase B to achieve a temperature of 70 degree Celsius; and
g) adding phase A into phase B followed by addition of 0.50% w/w of phenoxyethanol (and) ethylhexylglycerin to obtain the sunscreen composition.

25. Use of a sunscreen composition for anti-erythema, the composition comprising:
a) a soluble melanin in a range of 1% to 30% w/w;
b) nano lignin in a range of 1% to 50% w/w; and
c) one or more of excipient selected from the group comprising of a solubility enhancer as sodium bicarbonate in a range of 0.5% to 2.5% w/w of the composition, a humectant as propylene glycol in a range of 1% to 5% w/w of the composition, an emollient as octyldodecyl oleate (and) octyldodecyl stearoyl stearate (and) polyhydroxystearic acid (and) octyldodecanol in range of 1% to 50% w/w of the composition, a dispersing agent as polyglyceryl-4 oleate (and) polyglyceryl-6 oleate (and) polyhydroxystearic acid in a range of 0.1% to 5% w/w of the composition; an emulsifier as octyldodecanol (and) octyldodecyl xyloside (and) PEG-30 dipolyhydroxystearate in a range of 1% to 5% w/w of the composition, a bodying agent as ceto stearyl alcohol in a range of 1% to 10% w/w of the composition and a preservative as phenoxyethanol (and) ethylhexylglycerin in a range of 0.5% to 1.11% w/w of the composition.

26. A sunscreen composition with SPF 30, comprising:
a) a phase A comprising soluble melanin in a range of 1% to 30% w/w and a first excipient;
b) a phase B comprising nano lignin in a range of 1% to 50% and a second excipient; and
c) one or more third excipient.

27. The sunscreen composition with SPF 30 as claimed in claim 26, wherein the first excipient is selected from a group consisting of a diluent, humectant or a mixture thereof.

28. The sunscreen composition with SPF 30 as claimed in claim 26, wherein the second excipient is selected from a group consisting of a dispersing agent, emulsifier, bodying agent, humectant, emollient, or a mixture thereof.

29. The sunscreen composition with SPF 30 as claimed in claim 26, wherein the third excipient is a preservative selected from a group consisting of phenoxyethanol (and) ethylhexylglycerin, DMDM hydantoin (and) methylchloroisothiazolinone (and) methylisothiazolinone, phenoxyethanol, caprylyl glycol, sodium benzoate, potassium sorbate or a mixture thereof.

30. A sunscreen gel with SPF 55, comprising:
a) a phase A comprising soluble melanin in a range of 1% to 50% w/w and a first excipient wherein the first excipient is selected from a group consisting of a diluent, humectant or a mixture thereof;
b) a phase B comprising xanthan gum in a range of 0.1% to 3% and a second excipient wherein the second excipient is selected from a group consisting of a dispersing agent, emulsifier, bodying agent, humectant, emollient, or a mixture thereof; and
c) one or more third excipient wherein the third excipient is a preservative selected from a group consisting of phenoxyethanol (and) ethylhexylglycerin, DMDM hydantoin (and) methylchloroisothiazolinone (and) methylisothiazolinone, phenoxyethanol, caprylyl glycol, sodium benzoate, potassium sorbate or a mixture thereof.
